(19) United States
(12) Patent Application Publication
(10) Pub. No.: US 2004/0096406 A1
(43) Pub. Date: May 20, 2004

(54) Composition containing ascorbic acid compound and screening agent, method of use

(75) Inventor: Valerie De Poilly, Fontenay Sous Bois (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/665,821

(22) Filed: Sep. 22, 2003

Related U.S. Application Data
(60) Provisional application No. 60/418,347, filed on Oct. 16, 2002.

(30) Foreign Application Priority Data
Sep. 30, 2002 (FR) 0212079

(51) Int. Cl. A61K 7/42

(52) U.S. Cl. 424/59

(57) ABSTRACT

The present invention relates to a composition containing at least one metal salt of phosphorylated ascorbic acid, at least one screening agent having at least one sulphonic function, and at least one maleic anhydride polymer. The composition is useful for, e.g., depigmenting the skin, for preventing and/or combating skin marks, wrinkles and/or fine lines on the skin, and for preventing and/or combating the signs of ageing of the skin. The invention also relates to the use of a maleic anhydride polymer to obtain a homogeneous aqueous mixture of a metal salt of phosphorylated ascorbic acid and of sulphonic UV-screening agent.

### REFERENCE TO PRIOR APPLICATIONS

This application claims priority to U.S. provisional application 60/418,347, filed Oct. 16, 2002, and to French patent application 0212079 filed Sep. 30, 2002, both incorporated herein by reference.

### FIELD OF THE INVENTION

The present invention relates to a composition comprising at least one metal salt of phosphorylated ascorbic acid, at least one UV-screening agent comprising a sulphonic function, and at least one maleic anhydride polymer, and to its uses for example in cosmetics and dermatology, especially for depigmenting the skin, for preventing and/or combating skin marks, wrinkles and/or fine lines on the skin, and for preventing and/or combating the signs of ageing of the skin. Preferably the composition is an aqueous composition, and preferably comprises a physiologically acceptable medium.

The invention also relates to the use of a maleic anhydride polymer to obtain a homogeneous aqueous mixture of a metal salt of phosphorylated ascorbic acid and of sulphonic UV-screening agent.

### BACKGROUND OF THE INVENTION

It is known practice to introduce into cosmetic compositions various active agents intended to provide the skin and/or the hair with specific treatments. However, some of these active agents have the drawback of being unstable in aqueous medium and of readily degrading on contact with water, in particular on account of oxidation phenomena. They thus rapidly lose their activity over time, and this instability runs counter to the desired efficacy.

It has thus been sought for a long time to formulate ascorbic acid or vitamin C, on account of its numerous beneficial properties. In particular, ascorbic acid stimulates the synthesis of connective tissue and especially of collagen, reinforces the defenses of cutaneous tissue against external attacking factors such as ultraviolet radiation and pollution, compensates for the deficiency of vitamin E in the skin, depigments the skin and has free-radical-scavenging activity. These last two properties make it an excellent candidate as a cosmetic or dermatological active agent for combating or preventing ageing of the skin. Unfortunately, on account of its α-keto lactone chemical structure, ascorbic acid is very sensitive to certain environmental parameters and especially to oxidation phenomena. This therefore results in a rapid degradation of ascorbic acid formulated in the presence of these parameters and more particularly in the presence of oxygen, light or metal ions, as a function of temperature, or under certain pH conditions (Pharm. Acta. Helv., 1969, 44, 611-667; STP Pharma, 1985, 4, 281-286).

Several solutions have thus been envisaged in the prior art for reducing and/or delaying the degradation of ascorbic acid. One of these solutions consisted in using ascorbic acid derivatives. Examples of ascorbic acid derivatives that may be mentioned include the metal salts of phosphorylated ascorbic acid and especially magnesium ascorbyl phosphate.

Moreover, molecules capable of helping cell to defend themselves against an excess of photoinduced free radicals, and especially sunscreens (or UV-screening agents), which are capable of protecting the skin against attack caused by sunlight and ultraviolet light, are frequently combined with such active agents. Some of these sunscreens that are particularly effective are water-soluble compounds containing a sulphonic function, and it has been observed that it is very difficult to obtain a stable and uniform composition when the composition contains such a screening agent and a metal salt of phosphorylated ascorbic acid.

Thus, there is still a need for a cosmetic and/or dermatological composition which shows good stability although containing both metal salts of phosphorylated ascorbic acid and UV-screening agents comprising a sulphonic function.

### DETAILED DESCRIPTION OF THE PREFERRED EMBODIMENTS

The inventors have discovered, surprisingly, that whereas the usual chelating agents such as EDTA (ethylenediaminetetraacetic acid) and the salts thereof are ineffective for solving the problem of the invention, the use of a particular polymer in a composition containing one or more metal salts of phosphorylated ascorbic acid and one or more screening agents comprising at least one sulphonic function makes it possible to solve the problem of the incompatibility of these compounds, and to obtain uniform compositions.

Thus, one subject of the present invention is a composition comprising, preferably in a topically acceptable medium or a physiologically acceptable medium, at least one metal salt of phosphorylated ascorbic acid, at least one water-soluble UV-screening agent comprising at least one sulphonic function, and at least one maleic anhydride polymer. Preferably, the composition is an aqueous composition.

The expression “topically acceptable medium” means a medium that is compatible with cutaneous tissues such as the skin, the scalp, the eyelashes, the eyebrows, the hair, the nails and mucous membranes. The composition of the invention is preferably intended for topical application, and may be in the form of a cosmetic or dermatological composition. The present invention may also preferably comprise a physiologically acceptable medium.

While not bound by any theory, it is believed that it is the polymer that allows the production of a uniform aqueous composition of a metal salt of phosphorylated ascorbic acid and of a water-soluble UV-screening agent comprising at least one sulphonic function.

Thus, a subject of the invention is also the use of a maleic anhydride polymer to obtain a uniform aqueous composition comprising at least one metal salt of phosphorylated ascorbic acid and at least one water-soluble UV-screening agent comprising at least one sulphonic function.

The term “uniform” means a composition not comprising crystals and having a smooth appearance to the naked eye.

Maleic Anhydride Polymers

According to the invention, the term “maleic anhydride polymer” means both maleic anhydride homopolymers and copolymers and, in general, any polymer obtained by polymerization or copolymerization of one or more maleic anhydride units, these units optionally being partially or totally hydrolysed. Preferably, hydrophilic polymers are used in the composition of the invention, i.e. polymers with a water solubility of greater than or equal to 2 g/l.

Polymers that are particularly suitable for performing the invention include polymers obtained by polymerization or copolymerization of one or more maleic anhydride units, the maleic anhydride units of which are in hydrolysed form and preferably in the form of alkaline salts, for example in the form of sodium, potassium or lithium salts, the sodium salts being preferred.

In one advantageous aspect of the invention, the polymers are in the form of copolymers comprising maleic anhydride monomers and comonomers chosen from one or more of vinyl acetate, vinyl alcohol, vinylpyrrolidone, olefins containing from 2 to 20 carbon atoms, for instance octadecene, ethylene isobutylene, diisobutylene, isooctylene and alkyl vinyl ethers, in particular methyl vinyl ether or stearyl vinyl ether, and styrene, and mixtures thereof.

In one advantageous aspect of the invention, the polymer used according to the invention has a mole fraction of maleic anhydride units of between 0.1 and 1 and more preferably between 0.4 and 0.9.

The weight-average molar mass (weight) of the maleic anhydride polymers used according to the invention is not limited, but is preferably 1000 to 500,000 and preferably from 1,000 to 50,000 such as 2000, 5000, 10,000, 15,000, 20,000, 30,000, 40,000, etc.

According to one particular embodiment of the invention, the polymer used is a copolymer of styrene and of maleic anhydride and especially a copolymer of styrene and of maleic anhydride in a 50/50 ratio.

Useful polymers that are particularly suitable include the styrene/maleic anhydride copolymer (50/50) (CTFA name: sodium styrene/maleic acid copolymer) in sodium salt form at 40% in water, sold under the reference SMA1000HNa® by the company Atofina.

The amount of polymer present in the composition according to the invention is not particularly limited. Preferably, the polymer is present in an amount that is sufficient to obtain the desired effect, i.e. in an amount that is sufficient for the mixture of the metal salt of phosphorylated ascorbic acid and of screening agent containing a sulphonic function to be stable and uniform. According to one particular embodiment of the invention, the molar ratio between the amount of maleic anhydride units and the metal salt of phosphorylated ascorbic acid ranges from 0.005 to 10 and preferably from 0.01 to 1.

Preferably, the amount of polymer (as active material) ranges from 0.05% to 30% by weight and more particularly from 0.1% to 10% by weight relative to the total weight of the composition, including 1, 3, 5, 12, 14, 16, 18% by weight etc.

Metal Salt of Phosphorylated Ascorbic Acid

The metal salt of phosphorylated ascorbic acid may be chosen from alkali metal ascorbyl phosphates, alkaline-earth metal ascorbyl phosphates, transition metal ascorbyl phosphates, and mixtures thereof. Preferred materials include magnesium, sodium, potassium, calcium or zinc ascorbyl phosphate, and mixtures thereof. According to one preferred embodiment of the invention, the salt is magnesium ascorbyl phosphate.

The amount of metal salt of phosphorylated ascorbic acid is not particularly limited and can vary within a wide range depending on the desired aim. This amount may range, for example, from 0.1% to 20% by weight relative to the total weight of the composition, more particularly from 0.05% to 10% by weight and better still from 0.05% to 5% by weight relative to the total weight of the composition, including 0.1, 0.5, 1, 2, 3, 4, 6, 8% etc.

UV-Screening Agents

The screening agents used in the composition of the invention contain at least one sulphonic function and are water-soluble. They are preferably chosen from water-soluble sulphone and/or sulphonate screening agents. These screening agents may be partially neutralized with an organic base, for instance triethanolamine or ethylenediamine.

The screening agents used may be chosen especially from sulphone or sulphonate derivatives of benzylidenecamphor, of benzophenone or of phenylbenzimidazole, and mixtures thereof.

According to one preferred embodiment of the invention, the sulphone or sulphonate screening agents used in the invention are benzylidenecamphor derivatives.

Particularly preferred benzylidenecamphor derivatives that may be used in the invention have the general formula a):

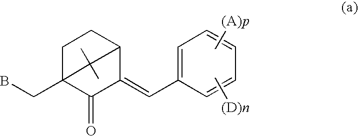

in which:

B represents —H or —SO₃H,

0≦p≦1 with B=—SO₃H when p=0,

0≦n≦4,

D represents one or more linear or branched alkyl or alkoxy radicals, which may be identical or different when n≧2, containing from 1 to 18 carbon atoms approximately, a halo radical or a hydroxyl radical.

A, which is preferably in the meta or para position, represents:

either an SO₃H radical;

or a group:

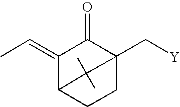

in which Y represents H or SO₃H;

or a group:

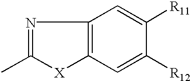

in which:

R₁₁ denotes a hydrogen atom, a linear or branched alkyl or alkoxy radical containing from 1 to 6 carbon atoms approximately or an —SO₃H radical, R₁₁ being —SO₃H when B=—H,

R₁₂ denotes a hydrogen atom or a linear or branched alkyl or alkoxy radical containing from 1 to 6 carbon atoms approximately,

X is an oxygen or sulphur atom or a group —NR—, R being a hydrogen atom or a linear or branched alkyl radical containing from 1 to 6 carbon atoms approximately,

and in which at least one —SO₃H function is optionally neutralized.

Particular examples of compounds of formula (a) that may be preferably used herein include the derivatives of formulae (I), (II) and (III) below:

Formula (I):

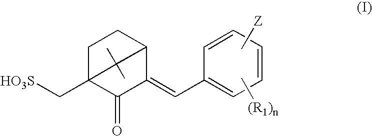

in which:

Z, preferably in the para or meta position, denotes a group

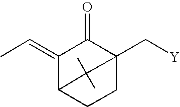

in which Y represents —H or —SO₃H, which is optionally neutralized,

n is equal to 0 or is a number ranging from 1 to 4 (0≦n≦4),

R₁ represents one or more linear or branched, identical or different alkyl or alkoxy radicals preferably containing from about 1 to 4 carbon atoms.

A compound of formula (I) that is particularly preferred is the compound corresponding to n=0, Z is in the para position and Y=—SO₃H: benzene-1,4-bis(3-methylidenecamphor-10-sulphonic acid), also known as terephthalylidenedicamphorsulphonic acid or (according to the CTFA nomenclature) “Terephthalylidene Dicamphor Sulphonic Acid” and manufactured under the name “Mexoryl SX” by the company Chimex.

Formula (II):

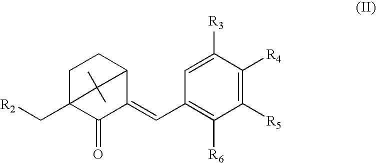

in which:

R₂ denotes a hydrogen atom or an —SO₃H radical,

R₃, R₄, R₅ and R₆, which may be identical or different, represent a hydroxyl group, a linear or branched alkyl radical containing from 1 to 4 carbon atoms approximately, a linear or branched alkenyl radical containing from 2 to 4 carbon atoms approximately, a linear or branched alkoxy radical containing from 1 to 4 carbon atoms, a linear or branched alkenyloxy radical containing from 2 to 4 carbon atoms, or a halo radical; furthermore, only one radical R₃ to R₆ may be an SO₃H radical, at least one of the radicals R₃ to R₆ denoting the SO₃H radical when R₂ is a hydrogen atom. One or more —SO₃H functions may also be neutralized.

Particular examples that may be mentioned include the following compounds of formula (II) in which:

R₄ denotes an —SO₃H radical in the para position of the benzylidenecamphor and R₂, R₃, R₅ and R₆ each denote a hydrogen atom, i.e. 4′-sulpho-3-benzylidenecamphor (CTFA name: Benzylidene Camphor Sulphonic Acid), sold under the name “Mexoryl SL” by the company Chimex,

R₃, R₃, R₅ and R₆ each denote a hydrogen atom and R₂ denotes an —SO₃H radical, i.e. 3-benzylidenecamphor-10-sulphonic acid,

R₄ denotes a methyl radical in the para position of the benzylidenecamphor, R₅ denotes an —SO₃H radical and R₂, R₃ and R₆ represent a hydrogen atom, i.e. 4′-methyl-3-benzylidenecamphor-3′-sulphonic acid,

R₄ denotes a chlorine atom in the para position of the benzylidenecamphor, R₅ denotes an —SO₃H radical and R₂, R₃ and R₆ represent a hydrogen atom, i.e. 4′-chloro-3-benzylidenecamphor-3′-sulphonic acid,

R₄ denotes a methyl radical in the para position of the benzylidenecamphor, R₃, R₅ and R₆ denote a hydrogen atom and R₂ denotes an —SO₃H radical, i.e. 4′-methyl-3-benzylidenecamphor-10-sulphonic acid,

R₂ represents an —SO₃H radical, R₃ is a methyl radical, R₄ is a hydrogen atom, R₅ is a tert-butyl radical and R₆ is a hydroxyl radical, i.e. (3-t-butyl-2-hydroxy-5-methyl)-3-benzylidenecamphor-10-sulphonic acid,

R₂ represents an —SO₃H radical, R₃ is a methoxy radical, R₄ is a hydrogen atom, R₅ is a tert-butyl radical and R₆ is a hydroxyl radical, i.e. (3-t-butyl-2-hydroxy-5-methoxy)-3-benzylidenecamphor-10-sulphonic acid,

R₂ represents an —SO₃H radical, R₃ and R₅ each denote a tert-butyl radical, R₄ denotes a hydroxyl radical and R₆ denotes a hydrogen atom, i.e. (3,5-ditertbutyl-4-hydroxy)-3-benzylidenecamphor-10-sulphonic acid,

R₄ represents a para-methoxy radical, R₅ represents —SO₃H and the radicals R₂, R₃ and R₆ represent H, i.e. 4′-methoxy-3-benzylidenecamphor-3′-sulphonic acid,

R₂ denotes an —SO₃H radical, R₃ and R₆ represent H, R₄ and R₅ forming a methylenedioxy radical, i.e. 3-(4,5-methylenedioxy)benzylidenecamphor-10-sulphonic acid,

R₂ represents an —SO₃H radical, R₄ represents a methoxy radical and the radicals R₃, R₅ and R₆ represent H, i.e. 3-(4-methoxy)benzylidenecarnphor-10-sulphonic acid,

R₂ represents an —SO₃H radical, R₄ and R₅ are both a methoxy radical and the radicals R₃ and R₆ represent H, i.e. 3-(4,5-dimethoxy)benzylidenecamphor-10-sulphonic acid,

R₂ represents an —SO₃H radical, R₄ is an n-butoxy radical and the radicals R₃, R₅ and R₆ represent a hydrogen atom, i.e. 3-(4-n-butoxy)benzylidenecamphor-10-sulphonic acid,

R₂ represents an —SO₃H radical, R₄ is an n-butoxy radical, R₅ is a methoxy radical and R₃ and R₆ both represent a hydrogen atom, i.e. 3-(4-n-butoxy-5-methoxy)benzylidenecamphor-10-sulphonic acid.

Formula (III):

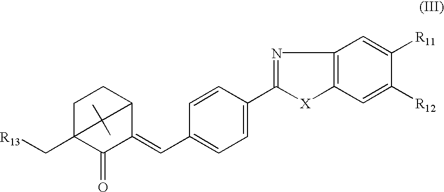

in which:

R₁₁ denotes a hydrogen atom, a linear or branched alkyl or alkoxy radical containing from 1 to 6 carbon atoms approximately or an —SO₃H radical,

R₁₂ denotes a hydrogen atom or a linear or branched alkyl or alkoxy radical containing from 1 to 6 carbon atoms approximately,

R₁₃ denotes a hydrogen atom or an —SO₃H radical,

at least one of the radicals R₁₁ and R₁₃ denoting an —SO₃H radical,

X is an oxygen or sulphur atom or a group —NR—, R being a hydrogen atom or a linear or branched alkyl radical preferably containing from 1 to 6 carbon atoms.

A particular example of a compound of formula (III) that may be mentioned is the compound in which X denotes an —NH— radical, R₁₁ denotes an —SO₃H radical and R₁₂ and R₁₃ both denote a hydrogen atom, i.e. 2-[4-(camphormethylidene)phenyl]benzimidazole-5-sulphonic acid.

The compounds of structures (I), (II) and (III) are described in documents U.S. Pat. No. 4,585,597, FR-A-2 236 515, FR-A-2 282 426, FR-A-2 645 148, FR-A-2 430 938 and FR-A-2 592 380, all incorporated herein by reference.

Further examples of benzylidenecamphor derivatives that may be used in the invention include the compounds of general formula (b) below:

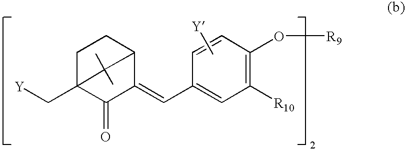

in which:

R₉ denotes a divalent radical: —(CH₂)ₘ— or —CH₂—CHOH—CH₂—, m being an integer ranging from 1 to 10 (1≦m≦10),

R₁₀ denotes a hydrogen atom, an alkoxy radical containing from 1 to 4 carbon atoms approximately or a divalent radical —O— linked to the radical R₉ when the said radical is also divalent,

Y and Y′ denote a hydrogen atom or an —SO₃H radical, at least one of these radicals Y or Y′ being other than hydrogen. In this case also, the —SO₃H function may be neutralized.

Particular examples that may be mentioned include the following compounds of formula (b) in which Y represents —SO₃H, Y′ is —H, R₁₀ is H and R₉ is —CH₂—CH₂—, i.e. ethylenebis[(4-oxybenzylidene)-3-camphor-10-sulphonic acid].

As another screening agent that may be used in the composition of the invention, mention may also be made of phenylbenzimidazolesulphonic acid (CTFA name: Phenylbenzimidazole Sulphonic Acid), sold under the trade name Eusolex 232 by the company Merck.

The amount of UV-screening agent(s) containing a sulphonic function used in the invention is not particularly limited and may vary within a wide range depending on the desired sun protection and the desired SPF for the composition. This amount (as active material) may range, for example, from 0.01% to 10% by weight relative to the total weight of the composition and more particularly from 0.02% to 5% by weight of active material relative to the total weight of the composition, including 0.1, 0.5, 1, 2, 3, 4, 6, 7, % etc.

Nonlimiting examples of compounds, materials, properties etc that may also be present in the invention composition will be described below.

The physiologically acceptable medium of the aqueous composition according to the invention preferably comprises water. It may more particularly consist of water and optionally of a physiologically acceptable organic solvent(s) chosen, for example, from lower alcohols containing from 1 to 8 carbon atoms and in particular from 1 to 6 carbon atoms, for instance ethanol, isopropanol, propanol or butanol; polyethylene glycols containing from 6 to 80 ethylene oxide units; polyols, for instance propylene glycol, isoprene glycol, butylene glycol, glycerol or sorbitol; and mixtures thereof.

The composition according to the invention generally preferably has a pH that is compatible with the skin, most preferably ranging from 2 to 7 and better still from 3 to 6, this pH varying depending on the acidic active agents contained in the composition.

The compositions according to the invention may be in any presentation form whatever, such as those conventionally used for topical application and especially in the form of aqueous or aqueous-alcoholic solutions, or aqueous gels, or, when an oily phase is added, in the form of oil-in-water (O/W) emulsions or water-in-oil (W/O) emulsions or multiple emulsions (triple emulsion: W/O/W or O/W/O), or dispersions of an oily phase in an aqueous phase using spherules, these spherules possibly being polymer nanoparticles such as nanospheres and nanocapsules, or lipid vesicles of ionic and/or nonionic type (liposomes, niosomes or oleosomes). These compositions are prepared according to the usual methods.

In addition, the compositions according to the invention may be more or less fluid and may have the appearance of a white or coloured cream, an ointment, a milk, a lotion, a serum, a paste or a mousse. They may optionally be applied to the skin in the form of an aerosol. They may also be in solid form, for example in the form of a stick.

When the composition according to the invention comprises an oily phase, this phase preferably contains at least one oil, especially a cosmetic oil. It may also contain other fatty substances.

Useful oils which can be used in the composition of the invention include:

hydrocarbon-based oils of animal origin, such as perhydrosqualene;

hydrocarbon-based oils of plant origin, such as liquid triglycerides of fatty acids containing from 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter;

synthetic esters and ethers, in particular of fatty acids, such as the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, branched-chain hydrocarbon-based oils comprising from 10 to 20 carbon atoms, such as isohexadecane, isododecane, isoparaffins and mixtures thereof, vaseline, polydecenes, hydrogenated polyisobutene such as Parléam® oil;

natural or synthetic essential oils such as, for example, eucalyptus oil, hybrid lavender oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;

fatty alcohols and fatty acids containing from 8 to 26 carbon atoms, such as cetyl alcohol or cetyl acid, stearyl alcohol, stearic acid, the mixture of cetyl alcohol and of stearyl alcohol (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2-295912;

silicone oils such as volatile or non-volatile polydimethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendent or at the end of a silicone chain, these groups containing from 2 to 24 carbon atoms; phenylsilicones such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

The term “hydrocarbon-based oil” in the list of the abovementioned oils embraces any oil comprising predominantly carbon and hydrogen atoms, and optionally ester, ether, fluoro, carboxylic acid and/or alcohol groups.

Other fatty substances which may be present in the oily phase include, for example, fatty acids containing from 8 to 30 carbon atoms, for instance stearic acid, lauric acid, palmitic acid and oleic acid; waxes, for example lanolin, beeswax, carnauba wax, candelilla wax, paraffin wax, lignite wax or microcrystalline waxes, ceresine or ozokerite, synthetic waxes, for instance polyethylene waxes and Fischer-Tropsch waxes; silicone resins such as trifluoromethyl-C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art in order to prepare a composition having the desired properties, for example consistency or texture properties, in view of this disclosure.

When the composition according to the invention is a water-in-oil (W/O) or oil-in-water (O/W) emulsion, the proportion of oily phase in the emulsion may preferably range from 5% to 80% by weight and more preferably from 5% to 50% by weight relative to the total weight of the composition.

The emulsions may contain at least one emulsifier chosen from amphoteric, anionic, cationic and nonionic emulsifiers, used alone or as a mixture, and optionally a co-emulsifier. The emulsifiers are chosen in an appropriate manner depending on the emulsion to be obtained (W/O or O/W). The emulsifier and the co-emulsifier are generally present in the composition in a proportion that can range, for example, from 0.3% to 30% by weight and preferably from 0.5% to 20% by weight relative to the total weight of the composition.

Examples of emulsifiers that may be mentioned for the W/O emulsions include dimethicone copolyols such as the mixture of cyclomethicone and of dimethicone copolyol, sold under the name “DC 5225 C” by the company Dow Corning, and alkyldimethicone copolyols, such as the laurylmethicone copolyol sold under the name “Dow Corning 5200 Formulation Aid” by the company Dow Corning, the cetyldimethicone copolyol sold under the name Abil EM 90® by the company Goldschmidt, or the mixture of cetyldimethicone copolyol, polyglyceryl-4 isostearate and hexyl laurate, sold under the name Abil WE09® by the company Goldschmidt. One or more co-emulsifiers may also be added thereto, which may be advantageously chosen from the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Surfactants of W/O emulsions that may also be used include a crosslinked elastomeric solid organopolysiloxane comprising at least one oxyalkylenated group, such as those obtained according to the procedure of Examples 3, 4 and 8 of document U.S. Pat. No. 5,412,004 and the examples of document U.S. Pat. No. 5,811,487, especially the product of Example 3 (synthesis example) of patent U.S. Pat. No. 5,412,004, such as the product sold under the reference KSG 21 by the company Shin Etsu, these patents being incorporated herein by reference.

Useful emulsifiers for the O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; the ethers of fatty alcohols; the sugar ethers of C₈-C₂₄ fatty alcohols, and mixtures thereof.

Glyceryl esters of fatty acids that may especially be mentioned include glyceryl stearate (glyceryl mono-, di- and/or tristearate) (CTFA name: glyceryl stearate) or glyceryl ricinoleate and mixtures thereof.

Polyethylene glycol esters of fatty acids that may especially be mentioned include polyethylene glycol stearate (polyethylene glycol mono-, di- and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

It is also possible to use mixtures of these surfactants, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (CTFA name: glyceryl stearate SE).

Fatty acid esters of glucose or of alkylglucose that may be mentioned in particular include glucose palmitate, alkylglucose sesquistearates, for instance methyl glucose sesquistearate, alkylglucose palmitates, for instance methylglucose palmitate or ethylglucose palmitate, fatty esters of methylglucoside and more especially the diester of methylglucoside and of oleic acid (CTFA name: Methyl glucose dioleate); the mixed ester of methylglucoside and of the oleic acid/hydroxystearic acid mixture (CTFA name: Methyl glucose dioleate/hydroxystearate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and of isostearic acid (CTFA name: Methyl glucose sesquiisostearate); the mixture of the monoester and diester of methylglucoside and of stearic acid (CTFA name: Methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate.

Examples of fatty alkyl ethers that may be mentioned include ethers of polyethylene glycol and of fatty alcohol containing from 8 to 30 carbon atoms and especially from 10 to 22 carbon atoms, such as the polyethylene glycol ethers of cetyl, stearyl or cetearyl alcohol (mixture of cetyl alcohol and stearyl alcohol). Mention may be made, for example, of ethers containing from 1 to 200 and preferably from 2 to 100 oxyethylenated groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol 10 by the company Kao Chemicals, the product sold under the name Plantaren 2000 by the company Henkel, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Henkel, and also arachidyl glucoside, for example in the form of the mixture of arachidyl alcohol and behenyl alcohol and arachidyl glucoside, sold under the name Montanov 202 by the company SEPPIC, and mixtures thereof.

According to one preferred embodiment of the invention, the composition is in the form of O/W emulsions.

The compositions of the invention may also contain one or more adjuvants such as those that are common in cosmetics or dermatology. Nonlimiting examples of such adjuvants include gelling agents, active agents, preserving agents, antioxidants, fragrances, solvents, fillers, sunscreens (=UV-screening agents) other than those indicated above, dyestuffs, basic agents (triethanolamine, diethanolamine or sodium hydroxide) or acidic agents, and also lipid vesicles or any other type of vector (nanocapsules, microcapsules, etc.), and mixtures thereof. These adjuvants are used in the usual proportions in the cosmetics field, for example from 0.01% to 30% of the total weight of the composition, and, depending on their nature, they are introduced into the aqueous phase of the compositions or into an oily phase when this is present, or alternatively into vesicles or any other type of vector. These adjuvants and the concentrations thereof preferably are such that they do not modify the desired properties of the invention.

Thus, depending on the fluidity of the composition that it is desired to obtain, it is possible to incorporate into the composition one or more gelling agents, especially hydrophilic gelling agents, i.e. agents that are soluble or dispersible in water. Examples of hydrophilic gelling agents that may be mentioned include modified or unmodified carboxyvinyl polymers, such as the products sold under the names Carbopol (CTFA name: carbomer) and Pemulen (CTFA name: Acrylates/C10-30 alkyl acrylate cross polymer) by the company Goodrich; polyacrylamides; optionally crosslinked and/or neutralized 2-acrylamido-2-methylpropane sulphonic acid polymers and copolymers, for instance the poly(2-acrylamido-2-methylpropanesulphonic acid) sold by the company Hoechst under the name “Hostacerin AMPS” (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, for instance xanthan gum, guar gum, alginates and modified celluloses; and mixtures thereof. The amount of gelling agents depends on the desired aim. The amount of gelling agents may range, for example, from 0.01% to 10% and preferably from 0.5% to 5% by weight relative to the total weight of the composition.

Among the fillers that may be used in the composition of the invention, examples that may be mentioned, besides pigments, include silica powder; talc; polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging for example from 0 to 20% by weight and preferably from 1% to 10% by weight relative to the total weight of the composition.

Useful active agents that may be used in the composition of the invention include enzymes (for example lactoperoxidase, lipase, protease, phospholipase and cellulases); flavonoids; moisturizers such as protein hydrolysates; sodium hyaluronate; polyols, for instance glycerol, glycols, for instance polyethylene glycols, and sugar derivatives; antiinflammatories; procyannidol oligomers; vitamins, for instance vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine, depigmenting agents such as kojic acid, hydroquinone and caffeic acid; salicylic acid and its derivatives; α-hydroxy acids such as lactic acid and glycolic acid and derivatives thereof; retinoids such as carotenoids and vitamin A derivatives; hydrocortisone; melatonin; algal extracts, fungal extracts, plant extracts, yeast extracts or bacterial extracts; steroids; antibacterial active agents, for instance 2,4,4′-trichloro-2′-hydroxydiphenyl ether (or triclosan), 3,4,4′-trichlorocarbanilide (or triclocarban) and the acids indicated above, and especially salicylic acid and its derivatives; matting agents, for instance fibres; tensioning agents; ceramides, essential oils; and mixtures thereof; and any active agent that is suitable for the final aim of the composition.

Useful steroids include dehydroepiandrosterone (or DHEA), and also (1) its precursors and biological derivatives, in particular the salts and esters of DHEA, such as DHEA sulphate and salicylate, 7-hydroxy DHEA, 7-keto DHEA, 7-hydroxy and 7-keto DHEA esters, especially 3-β-acetoxy-7-oxo DHEA, and (2) its precursors and chemical derivatives, in particular sapogenins such as diosgenin or hecogenin, and/or derivatives thereof such as hecogenin acetate, and/or natural extracts containing them and especially extracts of Dioscorea plants, such as wild yam.

The organic UV-screening agents other than those containing a sulphonic function described above may be present in an amount of active material ranging from 0.01% to 20% by weight of active material, preferably from 0.1% to 15% by weight and better still 0.2% to 10% by weight relative to the total weight of the composition.

As examples of UV-A-active and/or UV-B-active organic screening agents that may be added to the composition of the invention, mention may be made, for example, of para-aminobenzoic acid derivatives; salicylic derivatives such as ethylhexyl salicylate sold under the trade name Neo Heliopan OS by Haarmann & Reimer; dibenzoylmethane derivatives such as butyl methoxydibenzoyldimethane sold especially under the trade name Parsol 1789 by Hoffmann La Roche; cinnamic derivatives such as ethylhexyl methoxycinnamate sold especially under the trade name Parsol MCX by Hoffmann La Roche; β,β′-diphenylacrylate derivatives such as octocrylene (2-ethylhexyl α-cyano-β,βdiphenylacrylate) sold under the trade name Uvinul N539 by the company BASF; benzophenone derivatives such as Benzophenone-1 sold under the trade name Uvinul 400 by BASF, Benzophenone-2 sold under the trade name Uvinul D50 by BASF, Benzophenone-3 or Oxybenzone, sold under the trade name Uvinul M40 by BASF, benzophenone-4 sold under the trade name Uvinul MS40 by BASF; benzylidenecamphor derivatives such as 4-methylbenzylidenecamphor sold under the trade name Eusolex 6300 by Merck; phenylbenzimidazole derivatives such as Benzimidazilate sold under the trade name Neo Heliopan AP by Haarmann & Reimer; triazine derivatives such as Anisotriazine sold under the trade name Tinosorb S by Ciba Geigy and ethylhexyltriazone sold especially under the trade name Uvinul T150 by BASF; phenylbenzotriazole derivatives such as Drometrizole Trisiloxane sold under the trade name Silatrizole by the company Rhodia Chimie; anthranilic derivatives such as menthyl anthranilate sold under the trade name Neo Heliopan MA by Haarmann & Reimer; imidazoline derivatives; benzalmalonate derivatives; and mixtures thereof.

The composition according to the invention can constitute a cosmetic or dermatological composition and it may be used in all applications of vitamin C and its derivatives. It may be used especially for depigmenting and/or bleaching the skin, preventing and/or combating skin marks, wrinkles and/or fine lines on the skin, for preventing and/or combating the signs of ageing of the skin and/or for combating the harmful effects of UV radiation. It can also make it possible to tonify, regenerate and/or smooth out fine lines on the skin, and/or reinforce skin tissues against environmental attacking factors. It may also be used for treating the age marks that appear on the skin with age.

Thus, one subject of the invention is also the use of the composition as defined above for depigmenting and/or bleaching the skin, for preventing and/or combating skin marks, wrinkles and/or fine lines on the skin, for preventing and/or combating the signs of ageing of the skin and/or for combating the harmful effects of UV radiation.

A subject of the invention is also a treatment process for depigmenting and/or bleaching the skin, preventing and/or combating skin marks, wrinkles and/or fine lines on the skin, preventing and/or combating the signs of ageing of the skin and/or combating the harmful effects of UV radiation, comprising the application to the skin of a composition as defined above. Application may be site specific, if desired.

A preferred subject of the invention is also the use of the composition as defined above to manufacture an ointment for treating age marks.

The invention also relates to the use of one or more of the maleic anhydride polymers described above to obtain a homogeneous aqueous mixture of a metal salt of phosphorylated ascorbic acid and of sulphonic UV-screening agent. Such mixtures can be provided in any order of addition/contact including simultaneous addition/contact.

### EXAMPLES

The invention will now be illustrated by the following non-limiting examples. That is, the examples below of compositions, etc. according to the invention are given as illustration and with no limiting nature. The names are given as the CTFA names or as chemical names. The amounts therein are given as weight percentages, except where otherwise mentioned.

### Example 1 According to the Invention

| | |
|---|---|
| Water | 95.8% |
| Magnesium ascorbyl phosphate | 0.1% |
| Sodium styrene/maleic acid copolymer | 1% |
| (SMA 1000 HNa) | |
| Terephthalylidenedicamphorsulphonic acid | 3.1% |
| (raw material containing 33% active | |
| material in water) | |

(i.e. 1% active material)

The pH of the solution is 2.0. A clear solution is obtained.

### Comparative Example 2

| | |
|---|---|
| Water | 96.4% |
| Magnesium ascorbyl phosphate | 0.1% |
| Disodium EDTA | 0.4% |
| Terephthalylidenedicamphorsulphonic acid | 3.1% |
| (raw material containing 33% active | |
| material in water) | |

(i.e. 1% active material)

The pH of the solution is 2.0. A precipitate forms in the solution. This example shows that EDTA, known as a chelating agent, does not make it possible to solve the problem of the incompatibility between magnesium ascorbyl phosphate and the sulphonic screening agent.

### Comparative Example 3

| | |
|---|---|
| Water | 96.8% |
| Magnesium ascorbyl phosphate | 0.1% |
| Terephthalylidenedicamphorsulphonic acid | 3.1% |
| (raw material containing 33% active | |
| material in water) | |

(i.e. 1% active material)

The pH of the solution is 1.5. A precipitate forms in the solution. This example shows the incompatibility between magnesium ascorbyl phosphate and the sulphonic screening agent.

For Examples 1 to 3, the procedure is as follows:

The magnesium ascorbyl phosphate is placed in water, the polymer or the EDTA is added, followed by the UV-screening agent.

### Comparative Example 4

| | |
|---|---|
| Phase A: | |
| Glycerol | 3% |
| Methyl paraben (preserving agent) | 0.25% |
| Phenoxyethanol (preserving agent) | 0.5% |
| Water qs | 100% |
| Phase B: | |
| Xanthan gum | 0.2% |
| Ammonium polyacryloyldimethyltaurate | 1% |
| (Hostacerin AMPS) | |
| Acrylates/C10-30 alkyl acrylate | 0.2% |
| cross polymer | |
| Phase C: | |
| Triethanolamine | 0.2% |
| Phase D: | |
| Water | 2% |
| Terephthalylidenedicamphorsulphonic acid | 3.1% |
| (raw material containing 33% active | |
| material in water) | |

(i.e. 1% active material)

| | |
|---|---|
| Triethanolamine | 0.25% |
| Phase E: | |
| Dimethicone | 1% |
| Isononyl isononate | 1% |
| Cetearyl alcohol (and) Ceteareth-30 | 1% |
| (Emulgator E-2209 from the | |
| company Goldschmidt) | |
| Ethylhexyl methoxycinnamate | 1% |
| Acrylates copolymer | 0.25% |
| Cyclopentasiloxane | 0.5% |
| Phase F: | |
| Water | 2% |
| Magnesium ascorbyl phosphate | 0.1% |
| Tetrasodium EDTA | 0.2% |

Procedure: Phases A to F are successively mixed together by introducing them one after the other.

The composition obtained is not uniform and contains many crystals. This example shows that EDTA, which is known as a chelating agent, does not make it possible to solve the problem of the incompatibility between magnesium ascorbyl phosphate and the sulphonic screening agent.

### Example 5 According to the Invention (O/W Emulsion)

| | |
|---|---|
| Phase A: | |
| Glycerol | 3% |
| Methyl paraben (preserving agent) | 0.25% |
| Phenoxyethanol (preserving agent) | 0.5% |
| Water qs | 100% |
| Phase B: | |
| Xanthan gum | 0.2% |
| Ammonium polyacryloyldimethyltaurate | 1% |
| (Hostacerin AMPS) | |
| Acrylates/C10-30 alkyl acrylate cross polymer (Pemulen) | 0.2% |
| Phase C: | |
| Triethanolamine | 0.2% |
| Phase D: | |
| Water | 2% |
| Terephthalylidenedicamphorsulphonic acid (raw material | 3.1% |
| containing 33% active material in water) | |

(i.e. 1% active material)

| | |
|---|---|
| Triethanolamine | 0.25% |
| Phase E: | |
| Dimethicone | 1% |
| Isononyl isononate | 1% |
| Cetearyl alcohol (and) Ceteareth-30 | 1% |
| (Emulgator E-2209 from the | |
| company Goldschmidt) | |
| Ethylhexyl methoxycinnamate | 1% |
| Acrylates copolymer | 0.25% |
| Cyclopentasiloxane | 0.5% |
| Phase F: | |
| Water | 2% |
| Magnesium ascorbyl phosphate | 0.1% |
| Sodium styrene/maleic acid copolymer | 1% |
| (SMA 1000 HNa) | |

The composition obtained is fluid, smooth and uniform to the naked eye. It may be used as a photoprotective composition for treating and combating the signs of ageing of the skin (anti-ageing composition).

### Example 6 According to the Invention (O/W Emulsion)

| | |
|---|---|
| Phase A: | |
| Glycerol | 3% |
| Methyl paraben (preserving agent) | 0.25% |
| Phenoxyethanol (preserving agent) | 0.5% |
| Kojic acid | 1% |
| Water qs | 100% |
| Phase B: | |
| Xanthan gum | 0.2% |
| Ammonium polyacryloyldimethyltaurate | 1% |
| (Hostacerin AMPS) | |
| Acrylates/C10-30 alkyl acrylate | 0.2% |
| cross polymer (Pemulen) | |
| Phase C: | |
| Triethanolamine | 0.2% |
| Phase D: | |
| Water | 2% |
| Terephthalylidenedicamphorsulphonic acid | 3.1% |
| (raw material containing 33% active | |
| material in water) | |

(i.e. 1% active material)

| | |
|---|---|
| Triethanolamine | 0.25% |
| Phase E: | |
| Dimethicone | 1% |
| Isononyl isononate | 1% |
| Cetearyl alcohol (and) Ceteareth-30 | 1% |
| (Emulgator E-2209 from the | |
| company Goldschmidt) | |
| Ethylhexyl methoxycinnamate | 1% |
| Acrylates copolymer | 0.25% |
| Cyclopentasiloxane | 0.5% |
| Phase F: | |
| Water | 2% |
| Magnesium ascorbyl phosphate | 0.1% |
| Sodium styrene/maleic acid copolymer | 1% |
| (SMA 1000 HNa) | |

The composition obtained is fluid, smooth and uniform to the naked eye. It may be used as a depigmenting and photoprotective composition, for combating the appearance of marks.

The above written description of the invention provides a manner and process of making and using it such that any person skilled in this art is enabled to make and use the same, this enablement being provided in particular for the subject matter of the appended claims (e.g., a composition comprising water, at least one metal salt of phosphorylated ascorbic acid, at least one water-soluble UV-screening agent comprising at least one sulphonic function, and at least one maleic anhydride polymer) and further including an aqueous composition having a physiologically acceptable medium and comprising at least one metal salt of phosphorylated ascorbic acid, at least one water-soluble UV-screening agent comprising at least one sulphonic function, and at least one maleic anhydride polymer. Preferred methods/uses enabled herein include the cosmetic use of such a composition for depigmenting and/or bleaching the skin, preventing and/or combating skin marks, wrinkles and/or fine lines on the skin, for preventing and/or combating the signs of ageing of the skin and/or for combating the harmful effects of UV radiation, for treating the skin, for the manufacture of an, e.g., ointment for treating age marks. Preferred embodiments of the invention similarly fully described and enabled include the use of a maleic anhydride polymer to obtain a uniform aqueous composition containing at least one metal salt of phosphorylated ascorbic acid and at least one water-soluble UV-screening agent comprising at least one sulphonic function, for example by combining water, at least one maleic anhydride polymer, at least one metal salt of phosphorylated ascorbic acid, and at least one water-soluble UV-screening agent.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

1. A composition comprising water, at least one metal salt of phosphorylated ascorbic acid, at least one water-soluble UV-screening agent comprising at least one sulphonic function, and at least one maleic anhydride polymer.

2. The composition according to claim 1, wherein the composition is a uniform composition.

3. The composition according to claim 1, wherein maleic anhydride units of the polymer are in hydrolysed form and in the form of alkaline salts.

4. The composition according to claim 1, wherein the polymer is a copolymer comprising maleic anhydride units and comonomer units selected from the group consisting of vinyl acetate, vinyl alcohol, vinylpyrrolidone, olefins containing from 4 to 12 carbon atoms, alkyl vinyl ethers, styrene, and mixtures thereof.

5. The composition according to claim 1, wherein the polymer has a mole fraction of maleic anhydride units of between 0.1 and 1.

6. The composition according to claim 1, wherein the polymer is in the form of the sodium salt.

7. The composition according to claim 1, wherein the polymer is a copolymer of styrene and of maleic anhydride.

8. The composition according to claim 7, wherein the copolymer of styrene and of maleic anhydride is in a 50/50 ratio.

9. The composition according to claim 1, wherein the molar ratio between the amount of maleic anhydride units in said polymer and the metal salt of phosphorylated ascorbic acid ranges from 0.005 to 10.

10. The composition according to claim 1, wherein the polymer is present in an amount ranging from 0.05% to 30% by weight relative to the total weight of the composition.

11. The composition according to claim 1, wherein the metal salt of phosphorylated ascorbic acid is selected from the group consisting of alkali metal ascorbyl phosphates, alkaline-earth metal ascorbyl phosphates, transition metal ascorbyl phosphates, and mixtures thereof.

12. The composition according to claim 1, comprising magnesium ascorbyl phosphate.

13. The composition according to claim 1, wherein the metal salt of phosphorylated ascorbic acid is present in an amount ranging from 0.1% to 20% by weight relative to the total weight of the composition.

14. The composition according to claim 1, wherein the screening agent is selected from the group consisting of sulphone- or sulphonate-containing benzylidenecamphor, benzophenone, and phenylbenzimidazole compounds, and mixtures thereof.

15. The composition according to claim 1, comprising a benzylidenecamphor derivative of formula (I): 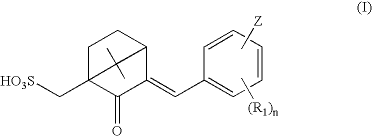
in which:
Z denotes a group 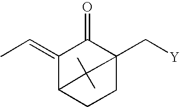
in which Y represents —H or —SO₃H, which is optionally neutralized,
n is equal to 0 or is a number ranging from 1 to 4,
R₁ represents one or more linear or branched, identical or different alkyl or alkoxy radicals containing from 1 to 4 carbon atoms.

16. The composition according to claim 1, comprising benzene-1,4-bis(3-methylidenecamphor-10-sulphonic acid).

17. The composition according to claim 1, comprising a benzylidenecamphor derivative of formula (II): 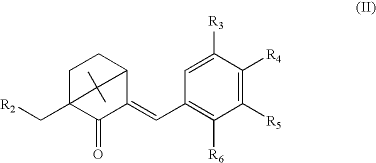
in which:
R₂ denotes a hydrogen atom or an —SO₃H radical,
R₃, R₄, R₅ and R₆, which may be identical or different, represent a hydroxyl group, a linear or branched alkyl radical containing from 1 to 4 carbon atoms approximately, a linear or branched alkenyl radical containing from 2 to 4 carbon atoms, a linear or branched alkoxy radical containing from 1 to 4 carbon atoms, a linear or branched alkenyloxy radical containing from 2 to 4 carbon atoms, or a halo radical; furthermore, only one radical R₃ to R₆ may be an —SO₃H radical, at least one of the radicals R₃ to R₆ denoting the —SO₃H radical when R₂ is a hydrogen atom.

18. The composition according to claim 1, comprising a benzylidenecamphor derivative of formula (III): 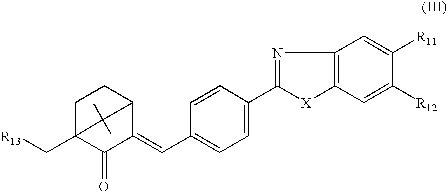
in which:
R₁₁ denotes a hydrogen atom, a linear or branched alkyl or alkoxy radical containing from 1 to 6 carbon atoms or an —SO₃H radical,
R₁₂ denotes a hydrogen atom or a linear or branched alkyl or alkoxy radical containing from 1 to 6 carbon atoms,
R₁₃ denotes a hydrogen atom or an —SO₃H radical,
at least one of the radicals R₁₁ and R₁₃ denoting an —SO₃H radical,
X is an oxygen or sulphur atom or a group —NR—, R being a hydrogen atom or a linear or branched alkyl radical containing from 1 to 6 carbon atoms.

19. The composition according to claim 1, wherein the amount of screening agent(s) ranges from 0.01% to 10% by weight relative to the total weight of the composition.

20. The composition according to claim 1, wherein said composition is in the form of an emulsion.

21. The composition according to claim 1, wherein said composition is in the form of a cosmetic or dermatological composition.

22. A method for depigmenting and/or bleaching the skin, preventing and/or combating skin marks, wrinkles and/or fine lines on the skin, for preventing and/or combating the signs of ageing of the skin and/or for combating the harmful effects of UV radiation, comprising applying to the skin an effective amount of the composition of claim 1.

23. A method for treating age marks, comprising applying to the skin an effective amount of the composition of claim 1.

24. The composition according to claim 1, wherein said composition further comprises a physiologically acceptable medium.

25. The composition according to claim 1, wherein said composition further comprises a topically acceptable medium.

26. The composition according to claim 1, wherein said composition is in the form of an O/W emulsion.

27. A method for preparing a uniform aqueous composition comprising at least one metal salt of phosphorylated ascorbic acid and at least one water-soluble UV-screening agent comprising at least one sulphonic function, comprising combining water, at least one maleic anhydride polymer, at least one metal salt of phosphorylated ascorbic acid, and at least one water-soluble UV-screening agent.
